(19) United States
(12) Patent Application Publication
(10) Pub. No.: US 2012/0114583 A1
(43) Pub. Date: May 10, 2012

(54) USE OF DIHYDROQUERCETIN AND AT LEAST ONE AMINO ACID TO POSITIVELY INFLUENCE THE NATURAL PIGMENTATION PROCESS

(75) Inventors: Melanie Giesen, Geldern (DE); Erik Schulze zur Wiesche, Hamburg (DE)

(73) Assignee: Henkel AG & Co. KGaA, Dusseldorf (DE)

(21) Appl. No.: 13/354,450

(22) Filed: Jan. 20, 2012

Related U.S. Application Data
(63) Continuation of application No. PCT/EP2010/060030, filed on Jul. 13, 2010.

(30) Foreign Application Priority Data
Jul. 23, 2009 (DE) 10 2009 027 960.1
Sep. 24, 2009 (DE) 10 2009 044 974.4

(51) Int. Cl. A61K 8/49; A61Q 5/00; A61K 8/67; A61K 8/60

(52) U.S. Cl. 424/70.1

(57) ABSTRACT

The present invention is a hair treatment agent comprising a combination of dihydroquercetin and/or a dihydroquercetin derivative with at least one amino acid. A preferred hair treatment agent comprises a combination of dihydroquercetin (taxifolin) with a six-amino acid mixture consisting of taurine, proline, valine, arginine, lysine, and glycine. The inventive hair treatment agent positively influences the natural pigmentation process of skin and skin appendages, such as for example, stimulating melanogenesis and pigmentation of hair, preventing and reducing the graying of hair, and repigmenting gray hair.

### CROSS-REFERENCE TO RELATED APPLICATIONS

This application is a continuation of PCT Application Serial No. PCT/EP2010/060030, filed on Jul. 13, 2010, which claims priority under 35 U.S.C. §119 to 10 2009 027 960.1 (DE), filed on Jul. 23, 2009 and to 10 2009 044 974.4 (DE) filed on Sep. 24, 2009. The disclosures PCT/EP2010/060030, DE 10 2009 027 960.1, and DE 10 2009 044 974.4 are hereby incorporated by reference in their entirety.

### FIELD OF THE INVENTION

The present invention relates to the use of a combination of dihydroquercetin, and/or a dihydroquercetin derivative, and at least one amino acid to positively influence the natural pigmentation process of skin and/or skin appendages.

### BACKGROUND OF THE INVENTION

In addition to its intrinsic physiological function, such as heat insulation and light protection, hair has a psychosocial function that must not be underestimated. Among other things it provides a means of interpersonal communication and represents a symbol of one's individuality. Changes, such as graying for example, can be enormously damaging to the self-confidence of the person concerned.

Hitherto, rather than combating the causes of hair graying, the hair has been treated with chemical colors that are often aggressive and hence damaging to the hair in order to cover the gray. Moreover, customers frequently complain of a lack of tolerance (itching, burning, prickling) and sustainability (affected areas have to be recolored at regular intervals). The effectiveness of the few biological products currently available on the market has not been scientifically proven and is often doubtful. Significantly effective, biologically active ingredients that influence the graying process directly at the root are not in use.

Pigmentation in the hair follicle is controlled by a defined and complex set of molecular signals. As melanogenesis in grayed follicles is evidently influenced, it can be assumed that the function of parts of this network is modified in the grayed follicle. One consequence of this is the reduction of melanin synthesis, which leads to graying of the follicle. The complex set of molecular signals that influence melanogenesis include inter alia the expression of MCR1 (melanocortin receptor 1), gp100 and ckit. MCR1 and ckit are receptors that transmit key signals of melanogenesis into the cell's interior by binding their ligands alpha-melanocyte stimulating hormone and stem cell factor. Gp100 is a protein of the melanosomal membrane and also regulates other proteins of relevance to melanogenesis. As these parameters are of essential significance in hair follicle pigmentation, it is advantageous to influence these parameters if melanin synthesis in the hair follicle cells is to be maintained or reactivated by the application of a test formulation. Retaining the pigmentation and hence the youthfulness of the hair by means of appropriate active ingredient formulations is a challenge for cosmetic research.

Use of dihydroquercetin in cosmetics because of its antioxidative properties is known from the prior art. Furthermore, a negative effect on the natural pigmentation process of skin and/or skin appendages by inhibition of the tyrosinase activity necessary for melanin synthesis is discussed in the specialist literature.

EP1845935 B1 claims the use of silybin, silymonin, silandrin, silychristin, silydianin and isosilybin in dermatological compositions for the induction, restoration or stimulation of a pigmentation of the skin, body hair or head hair.

There is clearly an unmet need for active ingredients suitable for positively influencing the natural pigmentation process, in particular in the hair or hair follicle, without exhibiting the aforementioned disadvantages of the methods known in the prior art for positively influencing hair color or the degree of hair graying and the youthful appearance of the hair.

### SUMMARY OF THE INVENTION

It has now been surprisingly found that the combination of dihydroquercetin and/or a dihydroquercetin derivative with at least one amino acid can positively influence the natural pigmentation process of skin and/or skin appendages.

In an exemplary embodiment of the present invention, a hair tonic or lotion comprises: at least one monohydric alcohol; dihydroquercetin and/or a dihydroquercetin derivative; at least one amino acid; optionally a gelling agent; and, optionally at least one specific care enhancer.

In another exemplary embodiment of the present invention, a hair treatment agent comprises: (a) 0.1 to 90 wt. % of at least one monohydric alcohol from the group comprising ethanol, n-propanol, isopropanol, n-butanol; (b) 0 to 10 wt. % of at least one gelling agent; (c) dihydroquercetin and/or a dihydroquercetin derivative; and, (d) at least one amino acid.

In another exemplary embodiment of the present invention, a hair treatment agent comprises: (a) 0.1 to 90 wt. % of at least one monohydric alcohol from the group comprising ethanol, n-propanol, isopropanol, n-butanol; (b) 0 to 10 wt. % of at least one gelling agent; (c) dihydroquercetin and; and, (d) at least one amino acid selected from taurine, proline, valine, arginine, lysine and glycine.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Dihydroquercetin is a flavonoid (3,3′,4′,5,7-pentahydroxyflavanone), also known as taxifolin. Preferred dihydroquercetin derivatives have the pentahydroxyflavanone framework and are etherified or esterified at one, two or more hydroxyl groups. Particularly preferred dihydroquercetin derivatives are dihydroquercetin monomethyl ethers, dihydroquercetin dimethyl ethers and dihydroquercetin glycosides, in particular the glucosides, dihydroquercetin xylosides, dihydroquercetin rhamnosides or dihydroquercetin galactosides. The O-3 glycosides in which the hydroxyl group is glycosylated at position 3 are particularly preferred.

Dihydroquercetin and/or the dihydroquercetin derivative (also referred to hereafter as dihydroquercetins) are preferably obtained as extracts. Dihydroquercetin-containing extracts are preferably used. In particular, extracts of silymarin (milk thistle) containing dihydroquercetin are used.

The extracts of dihydroquercetin can be prepared with water and with polar or non-polar organic solvents and mixtures thereof, in the manner known to the person skilled in the art. Extracts that can be obtained by extraction with ethanol or water/ethanol mixtures and pressed juice are preferred.

According to a preferred embodiment dihydroquercetin and/or dihydroquercetin derivative is used in a cosmetic agent that contains dihydroquercetin and/or the dihydroquercetin derivative in a total amount from 0.000001 to 3 wt. %, preferably 0.00001 to 1 wt. %, particularly preferably 0.0001 to 0.1 wt. %, exceptionally preferably 0.0003 to 0.05 wt. %, relative in each case to the total weight of the agent.

The second component of the combination/agent for use according to the invention is at least one amino acid, in particular one or more amino acids.

Amino acids that can be used according to the invention derive from the group consisting of glycine, alanine, valine, leucine, isoleucine, phenylalanine, tyrosine, taurine, tryptophane, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-citrulline, L-theanine, 3′,4′-dihydroxy-L-phenylalanine (L-dopa), 5′-hydroxy-L-tryptophane, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, and mixtures thereof.

The at least one amino acid is preferably selected from taurine, proline, valine, arginine, lysine, and glycine.

According to a more preferred embodiment, an amino acid mixture with at least two, three, four, five, six or seven amino acids is used.

According to a most preferred embodiment, the amino acid mixture comprises at least two, three, four, five or six amino acids selected from taurine, proline, valine, arginine, lysine, and glycine.

The following combinations of two amino acids are particularly preferred: Taurine with proline; taurine and valine; taurine and arginine; taurine and lysine; taurine and glycine; proline and valine; proline and arginine; proline and lysine; proline and glycine; valine and arginine; valine and lysine; valine and glycine; arginine and lysine; arginine and glycine; proline and valine; proline and arginine; proline and lysine; proline and glycine; valine and arginine; valine and lysine; valine and glycine; arginine and lysine; and, arginine and glycine.

The following combinations of three amino acids are particularly preferred: Taurine with proline and valine; taurine with proline and arginine; taurine with proline and lysine; taurine with proline and glycine; taurine with valine and arginine; taurine with valine and lysine; taurine with valine and glycine; taurine with arginine and lysine; taurine with arginine and glycine; taurine with lysine and glycine; proline and valine and arginine; proline and valine and lysine; proline and valine and glycine; proline and arginine and lysine; proline and arginine and glycine; proline and lysine and glycine; valine with arginine and lysine; valine with arginine and glycine; valine with lysine and glycine; and, arginine with lysine and glycine.

The following combinations of four amino acids are particularly preferred: Taurine with proline and valine and arginine; taurine with proline and valine and lysine; taurine with proline and valine and glycine; taurine with valine and arginine and lysine; taurine with valine and arginine and glycine; taurine with arginine and lysine and glycine; proline and valine and arginine and lysine; proline and valine and arginine and glycine; and, valine and arginine and lysine and glycine.

The following combinations of five amino acids are particularly preferred: Taurine with proline and valine and arginine and lysine; taurine with proline and arginine and lysine and glycine; taurine with proline and valine and arginine and glycine; taurine with valine and arginine and lysine and glycine; and, proline and valine and arginine and lysine and glycine.

The following combination of six amino acids is particularly preferred: Taurine and proline and valine and arginine and lysine and glycine.

In a further embodiment the aforementioned combinations can additionally also contain one or more amino acids that are not selected from those that are particularly preferred. This then results in likewise preferred combinations with three, four, five, six, seven and more amino acids.

The aforementioned combination of the six preferred amino acids taurine and proline and valine and arginine and lysine and glycine is most particularly preferred.

According to a most particularly preferred embodiment the ratio of the amounts of amino acids to one another in the amino acid mixture is from 10:1 to 1:10, in particular from 5:1 to 1:5, preferably from 1:2 to 2:1.

The ratio of amino acids in the amino acid mixture is most particularly preferably approximately 1:1. The aforementioned combination of the six preferred amino acids taurine and proline and valine and arginine and lysine and glycine in the ratio 1:1:1:1:1:1 is extremely preferred.

According to a preferred embodiment the at least one amino acid or the amino acid mixture is used in a cosmetic agent that contains the at least one amino acid or the amino acid mixture in a total amount from 0.000001 to 5 wt. %, preferably 0.00001 to 1 wt. %, particularly preferably 0.0001 to 0.1 wt. %, exceptionally preferably 0.0005 to 0.05 wt. %, relative in each case to the total weight of the agent.

According to a preferred embodiment the ratio of the amount of dihydroquercetin and/or the dihydroquercetin derivative to the total amount of the at least one amino acid or the amino acid mixture is from 10:1 to 1:10, in particular from 5:1 to 1:5, preferably from 3:1 to 1:4.

Particularly preferred combinations in agents for use according to the invention are 0.000001 to 3 wt. %, preferably 0.00001 to 1 wt. %, particularly preferably 0.0001 to 0.1 wt. %, exceptionally preferably 0.0003 to 0.05 wt. % of dihydroquercetin (taxifolin) and 0.000001 to 5 wt. %, preferably 0.00001 to 1 wt. %, particularly preferably 0.0001 to 0.1 wt. %, exceptionally preferably 0.0005 to 0.05 wt. % of at least one amino acid selected from taurine, proline, valine, arginine, lysine, and glycine, relative in each case to the total weight of the agent.

Particularly preferred combinations in agents for use according to the invention are 0.000001 to 3 wt. %, preferably 0.00001 to 1 wt. %, particularly preferably 0.0001 to 0.1 wt. %, exceptionally preferably 0.0003 to 0.05 wt. % of dihydroquercetin (taxifolin) and 0.000001 to 5 wt. %, preferably 0.00001 to 1 wt. %, particularly preferably 0.0001 to 0.1 wt. %, exceptionally preferably 0.0005 to 0.05 wt. % of at least one amino acid mixture comprising the amino acids taurine and proline and valine and arginine and lysine and glycine, in particular in the ratio of amino acids to one another of 1:1:1:1:1:1, relative in each case to the total weight of the agent.

Surprisingly it has been found that the use of a combination of dihydroquercetin and/or a dihydroquercetin derivative with at least one amino acid is capable of positively influencing, in particular stimulating, the natural pigmentation process, in particular in the hair or hair follicle. The combination according to the invention induced both the gene expression of MCR-1 and that of ckit and gp100 in a synergistic manner. Furthermore, an increase in melanin synthesis was observed. In addition, the natural pigmentation process is positively influenced by increasing the available ATP content and the hepatocyte growth factor (HGF) in the hair follicles. The natural pigmentation process of skin and/or skin appendages can thus be influenced, in particular stimulated, by application of the combination according to the invention or of the agents used according to the invention. In particular, the natural pigmentation process of the hair or hair follicle or in the hair follicle can thus be influenced, in particular stimulated. The agents used according to the invention are suitable for stimulating and/or improving the pigmentation of the hair, stimulating melanogenesis, in particular in the hair follicle, preventing and/or reducing hair graying and repigmenting gray hair.

Within the meaning of the present invention the term “influencing the natural pigmentation process” is understood to mean the positive influencing of the natural coloring/coloration and/or pigmentation of the skin and/or skin appendages, in particular the stimulation of the natural, i.e. biological, pigmentation process in the skin and/or skin appendages, in particular hair or hair follicles.

Within the context according to the invention, skin and skin appendages are understood to be the skin, mucous membranes, hair and hair follicles, glands and nails, in particular the skin, mucous membranes, hair and hair follicles. The term skin is particularly preferably understood to be the skin excluding the mucous membranes. The term skin appendages is most particularly preferably understood to be the hair and hair follicles, preferably body hair, beard hair and head hair, most particularly preferably beard hair and head hair, most particularly preferably head hair and the corresponding hair follicles.

According to a preferred embodiment, the positive influencing of the natural pigmentation process is understood to be the positive influencing of at least one sub-step of the natural pigmentation process. This influencing relates in particular to the regulation of the molecular signals that influence the biological or natural pigmentation process.

The regulation of the biological or natural pigmentation process through gene regulation, i.e. regulation at an expression level, and/or enzyme regulation, i.e. regulation at an activity level, and/or regulation at a hormone level is preferred.

The regulation of melanogenesis, inter alia regulation of the gene expression of MCR1 (melanocortin receptor 1), gp100 and ckit, is particularly preferred. The regulation of tyrosinase, both of the gene expression of tyrosinase and regulation at an enzyme level, is moreover also encompassed.

According to a preferred embodiment the natural pigmentation process of the hair is influenced, in particular stimulated or prompted. In particular, influencing is understood to be the positive influencing, preferably the positive regulation (up-regulation or activation or prompting or increase) that leads to a stimulation of the natural, biological pigmentation process. Stimulation of melanogenesis in the human hair follicle, in particular of the head hair (the hair follicle located on the scalp/top of the head), is particularly preferred.

According to the invention the pigmentation process, in particular melanogenesis, of the skin and skin appendages, preferably of the hair or hair follicle, can be influenced. In particular, the natural pigmentation process, in particular melanogenesis, in mammals, particularly preferably in humans, can be influenced. The pigmentation process, preferably melanogenesis, of the human hair or human hair follicle, is preferably influenced.

According to the invention, stimulation of melanogenesis is understood to be the stimulation, increase, prompting or improvement of melanin synthesis in the melanocytes (preferably the melanocytes in the hair follicle). This is achieved for example by an increase in the gene expression of signal molecules such as MCR1 (melanocortin receptor 1), gp100 and ckit. According to a preferred embodiment, the positive influencing, preferably stimulation, of melanogenesis is achieved by the use according to the invention. In particular, melanogenesis is stimulated in the hair or hair follicle of the haired scalp and/or beard, in particular in humans.

Within the meaning of the present invention, stimulation of pigmentation is understood in particular to be the improvement, increase and/or stimulation of the transport of melanosomes into the keratinocytes surrounding the hair follicle and also the pigmentation of the individual hair, a selection of hairs, in particular an area of haired skin, in particular scalp, or of the entire head and/or beard hair, that is perceptible to the eye or by correspondingly suitable measuring methods.

In the context of a preferred embodiment, hair graying, in particular of human hair, is prevented, preferably substantially prevented, and/or reduced by the use according to the invention. Within the meaning of the present invention, hair graying is understood to mean both the visually perceptible graying of hair due to the mixing of white and pigmented hair and the pigment dilution in an individual hair, in other words the graying of an individual hair.

A prevention of hair graying occurs in particular in hair that is not yet grayed, whereas a reduction of hair graying can take place both in already grayed hair and in hair that is not yet grayed. In the one case, hair follicles in which melanogenesis does not function or no longer functions or does not function completely or is disrupted or reduced are prompted/stimulated to melanogenesis again, whereas in hair/hair follicles that are not grayed, a disruption, reduction or down-regulation of melanogenesis does not occur at all or occurs only to a lesser extent.

According to a further preferred embodiment, hair that is already grayed is repigmented by the use according to the invention of a combination of dihydroquercetin and/or a dihydroquercetin derivative with at least one amino acid.

According to a further particularly preferred embodiment the use according to the invention is a cosmetic use that is non-therapeutic.

In particular, the use according to the invention, which is aimed at hair graying, in particular non-pathological hair graying, arising from the natural aging process, is a purely cosmetic use that does not constitute treatment and/or prevention of a disease and hence is non-therapeutic.

According to a particular embodiment, the use according to the invention takes place topically, i.e. by application onto the skin and/or skin appendages, in particular facial and/or head hair, in particular head hair.

The cosmetic agents according to the invention exhibit improved caring effects on skin and hair. The positive effects are clearly pronounced on keratinic fibers in particular, such that preferred cosmetic agents according to the invention are hair treatment agents.

Hair treatment agents within the meaning of the present invention are for example hair coloring agents, bleaching agents, hair shampoos, hair conditioners, conditioning shampoos, hair sprays, hair rinses, hair masks, hair packs, hair tonics, permanent wave fixing solutions, hair coloring shampoos, hair coloring agents, hair fixing agents, hair setting agents, hair styling preparations, blow-drying lotions, styling mousses, hair gels, hair waxes or combinations thereof. Particularly preferred hair treatment agents have the characterizing feature that they are formulated as a shampoo, hair tonic, hair mask, hair rinse, hair mousse, hair fixing agent, hair spray, hair gel and/or hair coloring agent. These agents are particularly advantageous in view of the fact that for reasons of time and convenience the consumer often shies away from the use of multiple different agents and/or multiple application steps.

A further preferred group of ingredients of the agents used according to the invention are vitamins, provitamins or vitamin precursors. These are described below.

The compositions for use according to the invention may contain surfactants, such as cationic surfactants. Preferred surfactants, and the amounts in which they are contained in the compositions, are disclosed in DE 102009044974 on pages 9 to 19, incorporated herein by reference. Particularly preferred hair treatment agents in accordance with the present invention comprise, relative to their total composition weight, 0.05 to 7.5 wt. %, preferably 0.1 to 5 wt. %, particularly preferably 0.2 to 3.5 wt. % and in particular 0.25 to 2.5 wt. % of cationic surfactant(s) selected from the group consisting of quaternary ammonium compounds and/or esterquats and/or amido amines, wherein preferred cationic surfactant(s) is/are selected from alkyl trimethylammonium chlorides having preferably 10 to 18 carbon atoms in the alkyl residue and/or dialkyl dimethylammonium chlorides having preferably 10 to 18 carbon atoms in the alkyl residue and/or trialkyl methylammonium chlorides having preferably 10 to 18 carbon atoms in the alkyl residue and/or cetyl trimethylammonium chloride and/or stearyl trimethylammonium chloride and/or distearyl dimethylammonium chloride and/or lauryl dimethylammonium chloride and/or lauryl dimethyl benzylammonium chloride and/or tricetyl methylammonium chloride and/or Quaternium-27 and/or Quaternium-83 and/or N-methyl-N(2-hydroxyethyl)-N,N-(ditalgacyloxyethyl)ammonium methosulfate and/or N-methyl-N(2-hydroxyethyl)-N,N-(distearoyloxyethyl)ammonium methosulfate and/or N,N-dimethyl-N,N-distearoyloxyethyl ammonium chloride, N,N-di-(2-hydroxyethyl)-N,N-(fatty acid ester ethyl)ammonium chloride, and mixtures thereof.

As a further optional constituent, the agents in accordance with the present invention may contain 0.01 to 10 wt. % of at least one polymer from the group of cationic and/or amphoteric polymers. Preferred polymers, and the amounts in which they are contained in the present compositions, are disclosed in DE 102009044974 on pages 19 to 29, incorporated herein by reference.

A further preferred group of ingredients of the agents used according to the invention are vitamins, provitamins or vitamin precursors. These are described below.

The group of substances classified as vitamin A include retinol (vitamin A₁) and 3,4-didehydroretinol (vitamin A₂). β-Carotene is the retinol provitamin. Suitable vitamin A components according to the invention include for example vitamin A acid and esters thereof, vitamin A aldehyde, and vitamin A alcohol and esters thereof, such as palmitate and acetate. The agents used according to the invention preferably contain the vitamin A component in amounts from 0.05 to 1 wt. %, relative to the complete preparation.

The vitamin B group, or the vitamin B complex, includes inter alia vitamin B₁ (thiamine), vitamin B₂ (riboflavin), and vitamin B₃. Nicotinic acid and nicotinic acid amide (niacinamide) are often included under this term. Preferred is nicotinic acid amide, in amounts from 0.05 to 1 wt. % relative to the total weight of complete agent. Also preferred is vitamin B₅ (pantothenic acid, panthenol and pantolactone). Within the context of this group, panthenol and/or pantolactone is preferably used. Derivatives of panthenol that can be used according to the invention are the esters and ethers of panthenol as well as cationically derivatized panthenols. Individual representatives are for example panthenol triacetate, panthenol monoethyl ether and the monoacetate thereof as well as the cationic panthenol derivatives disclosed in WO 92/13829. The cited compounds of the vitamin B₅ type are preferably contained in amounts from 0.05 to 10 wt. %, relative to the complete agent. Amounts from 0.1 to 5 wt. % are particularly preferred. Vitamin B₆ (pyridoxine as well as pyridoxamine and pyridoxal) can also be used. Vitamin C (ascorbic acid) may be used in the agents according to the invention in amounts from 0.1 to 3 wt. %, relative to the complete agent, with the form of the palmitic acid ester, glucosides, or phosphates being preferred. Use in combination with tocopherols is also preferred. Vitamin E (tocopherols, in particular α-tocopherol) and derivatives thereof, which include in particular the esters such as acetate, nicotinate, phosphate and succinate, are preferably contained in amounts from 0.05 to 1 wt. %, relative to the complete agent. Vitamin F is understood to mean essential fatty acids, in particular linoleic acid, linolenic acid and arachidonic acid. Vitamin H is the name given to the compound (3aS,4S,6aR)-2-oxohexahydrothienol[3,4-d]-imidazole-4-valeric acid, although this is now more widely known by the trivial name biotin. Biotin is preferably contained in the agents of the present invention in amounts from 0.0001 to 1.0 wt. %, in particular in amounts from 0.001 to 0.01 wt. %.

Combinations of dihydroquercetin and taurine with tocopherols, in particular alpha-tocopherol, are particularly preferred. Combinations of dihydroquercetin and proline with tocopherols, in particular alpha-tocopherol, are also particularly preferred. Combinations of dihydroquercetin and valine with tocopherols, in particular alpha-tocopherol, are particularly preferred. Combinations of dihydroquercetin and arginine with tocopherols, in particular alpha-tocopherol, are particularly preferred. Combinations of dihydroquercetin and lysine with tocopherols, in particular alpha-tocopherol, are particularly preferred. Combinations of dihydroquercetin and glycine with tocopherols, in particular alpha-tocopherol, are particularly preferred. Combinations of dihydroquercetin and taurine, proline, valine, arginine, lysine and glycine with tocopherols, in particular alpha-tocopherol, are most particularly preferred.

Particularly preferred hair treatment agents according to the invention contain, relative to their weight, 0.0001 to 1 wt. %, preferably 0.001 to 0.5 wt. % and particularly preferably 0.005 to 0.1 wt. % of at least one ubiquinone and/or at least one ubiquinol and/or at least one derivative of these substances. Particularly preferred is coenzyme Q10 in an amount from 0.005 to 0.1 wt. %. As an alternative to or in addition to the preferred ubiquinones, the agents according to the invention may also contain plastoquinones (polyprenylated 2,3-dimethylbenzoquinone derivatives). Preferred agents according to the invention may contain 0.0002 to 4 wt. %, preferably 0.0005 to 3 wt. %, particularly preferably 0.001 to 2 wt. %, more preferably 0.0015 to 1 and in particular 0.002 to 0.5 wt. % of at least one plastoquinone. The prenyl side chain contains n prenyl units. Values for n from 1 to 20, preferably from 2 to 15 and in particular 5, 6, 7, 8, 9, 10 are preferred, wherein agents that are particularly preferably to be used contain a plastoquinone having n=9.

A combination of dihydroquercetin and at least one amino acid with coenzyme Q10 is particularly preferred.

Agents that are preferred for use according to the invention contain as a care substance, relative to their weight, 0.01 to 15 wt. %, preferably 0.025 to 12.5 wt. %, particularly preferably 0.05 to 10 wt %, more preferably 0.1 to 7.5 wt. %, and in particular 0.5 to 5 wt. %, of at least one 2-furanone derivative of the formula (I) and/or of the formula (II):

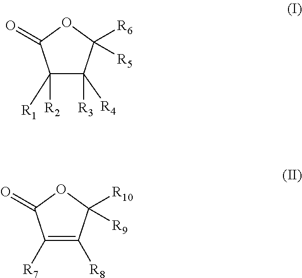

Suitable 2-furanone derivatives and the amounts in which they are contained in the compositions for use according to the invention are disclosed in DE 102009044974 on pages 34 to 39, incorporated herein by reference.

A further care substance that can preferably be used, and having activating properties, is taurine. Hair treatment agents that are preferred according to the invention contain as a care substance, relative to their weight, 0.01 to 15 wt. %, preferably 0.025 to 12.5 wt. %, particularly preferably 0.05 to 10 wt. %, more preferably 0.1 to 7.5 wt. % and in particular 0.5 to 5 wt. % of taurine (2-aminoethane sulfonic acid).

The agents used according to the invention may contain further ingredients that prevent, alleviate, or even cure hair loss. Active ingredients that stabilize the hair root are particularly advantageous. Propecia (finasteride) is currently the only preparation approved worldwide for which an effectiveness and tolerance has been proven in numerous studies. Propecia works by reducing the ability of DHT to form from testosterone. Minoxidil with or without supplementary additives is probably the oldest demonstrably effective hair growth agent. For the treatment of hair loss, Minoxidil should be used externally. There are hair lotions containing 2% to 5% minoxidil, also gels containing up to 15% minoxidil. The effectiveness increases with the dose, but in hair lotions minoxidil is soluble only in a content of up to 5%. In many countries hair lotions containing up to 2% minoxidil are available without a prescription. Spironolactone in the form of a hair lotion and in combination with minoxidil can be used for external application to combat hormonal influences on the hair follicles. Spironolactone works as an androgen receptor blocker, in other words binding of DHT to the hair follicles is prevented. Cosmetic agents for use according to the invention are particularly preferred which additionally contain, relative to their weight, 0.001 to 5 wt. % of hair root-stabilizing substances, in particular minoxidil and/or finasteride and/or ketoconazole.

The agents used according to the invention may contain any active ingredients, additives and auxiliary substances presently known for such preparations. In many cases the agents contain at least one surfactant, with anionic, zwitterionic, ampholytic, non-ionic, and cationic surfactants, all being suitable. It has proved advantageous in many cases, however, to select the surfactants from the group consisting of anionic, zwitterionic, and non-ionic surfactants. These surfactants have already been described in detail above.

A preferred physical form of the hair treatment agent according to the invention is in the form of hair tonics or hair lotions. These preferably contain at least one monohydric alcohol, dihydroquercetin and/or a dihydroquercetin derivative, at least one amino acid and optionally a gelling agent and optionally at least one specific care enhancer.

In a further embodiment the present invention provides a hair treatment agent comprising: (a) 0.1 to 90 wt. % of at least one monohydric alcohol from the group comprising ethanol, n-propanol, isopropanol, n-butanol; (b) 0 to 10 wt. % of at least one gelling agent; (c) dihydroquercetin and/or a dihydroquercetin derivative; and, (d) at least one amino acid.

A particularly preferred hair treatment agent comprises: (a) 0.1 to 90 wt. % of at least one monohydric alcohol from the group comprising ethanol, n-propanol, isopropanol, n-butanol; (b) 0 to 10 wt. % of at least one gelling agent; (c) dihydroquercetin and; and, (d) at least one amino acid selected from taurine, proline, valine, arginine, lysine and glycine.

The agents used according to the invention contain 0.1 to 90 wt. % of at least one monohydric alcohol from the group comprising ethanol, n-propanol, isopropanol, n-butanol, Of these, ethanol and/or isopropanol are particularly preferred. Particularly preferred hair treatment agents according to the invention have the characterizing feature that they contain, relative to their weight, 0.5 to 85 wt. %, preferably 1 to 80 wt. %, particularly preferably 5 to 75 wt. %, more preferably 10 to 70 wt. % and in particular 25 to 60 wt. % of ethanol and/or isopropanol.

Particularly preferred hair treatment agents contain exclusively ethanol. Hair treatment agents according to the invention that contain, relative to their weight, 5 to 80 wt. %, preferably 7.5 to 70 wt. %, particularly preferably 10 to 60 wt. %, more preferably 20 to 55 wt. % and in particular 25 to 50 wt. % of ethanol are particularly preferred here.

The agents used according to the invention can additionally contain a gelling agent. The adhesion of the agents to the hair can be improved and the application made more pleasant through the use of these gelling agents. Hair treatment agents according to the invention are preferred that, relative to their weight, contain 0.15 to 9 wt. %, preferably 0.2 to 8 wt. %, particularly preferably 0.25 to 7 wt. %, more preferably 0.3 to 6 wt. % and in particular 0.4 to 5 wt. % of at least one gelling agent from the groups of silicic acids and/or phyllosilicates and/or organophyllosilicates and/or metal soaps and/or hydrogenated castor oil and/or modified fat derivatives and/or polyamides and/or hydroxyethyl cellulose (HEC) and/or carboxymethyl cellulose (CMC) and/or hydroxypropyl methylcellulose (HPMC) and/or hydroxypropyl cellulose (HPC) and/or ethyl hydroxyethyl cellulose (EHEC) and/or polyvinyl alcohols and/or polyacrylic acid and/or polymethacrylic acids and salts thereof and/or polyacrylamides and/or polyvinyl pyrrolidone and/or polyethylene glycols and/or styrene-maleic anhydride copolymers and salts thereof and/or copolymers and/or terpolymers of acrylic acid and methacrylic acid and/or cellulose and/or starch and/or xanthan gum.

In a further preferred embodiment the agents used according to the invention may contain at least one emulsifier. Preferred emulsifiers, and the amounts in which they are contained in the compositions, are disclosed in DE 10 2009 044 974 on pages 42 to 43, incorporated herein by reference.

In a preferred embodiment of the invention, an agent according to the invention may also contain UV filters. There are no general restrictions on the UV filters to be used according to the invention in terms of their structure and their physical properties. Any UV filters that can be used in the cosmetics arts, and whose absorption maximum is in the UVA (315-400 nm), UVB (280-315 nm), or UVC (<280 nm) range, are suitable. UV filters having an absorption maximum in the UVB range, in particular in the range from approximately 280 to approximately 300 nm, are particularly preferred. The UV filters used according to the invention can be selected for example from substituted benzophenones, p-aminobenzoic acid esters, diphenyl acrylic acid esters, cinnamic acid esters, salicylic acid esters, benzimidazoles and o-aminobenzoic acid esters. Examples of UV filters that can be used according to the invention are 4-aminobenzoic acid, N,N,N-trimethyl-4-(2-oxobom-3-ylidene methyl)aniline methyl sulfate, 3,3,5-trimethyl cyclohexyl salicylate (homosalates), 2-hydroxy-4-methoxybenzophenone (Benzophenone-3; Uvinul®M 40, Uvasorb®MET, Neo Heliopan®BB, Eusolex®4360), 2-phenylbenzimidazole-5-sulfonic acid and potassium, sodium and triethanolamine salts thereof (Phenylbenzimidazole sulfonic acid; Parsol®HS; Neo Heliopan®Hydro), 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione (Butyl methoxydibenzoylmethane; Parsol®1789, Eusolex®9020), α-(2-oxobom-3-ylidene)toluene-4-sulfonic acid and salts thereof, ethoxylated 4-aminobenzoic acid ethyl ester (PEG-25 PABA; Uvinul®P 25), 4-dimethylaminobenzoic acid-2-ethylhexyl ester (Octyl Dimethyl PABA; Uvasorb®DMO, Escalol®507, Eusolex®6007), salicylic acid-2-ethylhexyl ester (Octyl Salicylate; Escalol®587, Neo Heliopan®OS, Uvinul®018), 4-methoxycinnamic acid isopentyl ester (Isoamyl p-Methoxycinnamate; Neo Heliopan®E 1000), 4-methoxycinnamic acid-2-ethylhexyl ester (Octyl Methoxycinnamate; Parsol®MCX, Escalol®557, Neo Heliopan®AV), 2-hydroxy-4-methoxybenzophenone-5-sulfonic acid and the sodium salt thereof (Benzophenone-4; Uvinul®MS 40; Uvasorb®S 5), 3-(4′-methylbenzylidene)-D,L-camphor (4-Methylbenzylidene camphor; Parsol®5000, Eusolex®6300), 3-benzylidene camphor (3-Benzylidene camphor), 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)[2-oxoborn-3-ylidene methyl]benzyl}acrylamide, 2,4-dihydroxybenzophenone (Benzophenone-1; Uvasorb®20 H, Uvinul®400), 1,1′-diphenylacrylonitrilic acid-2-ethylhexyl ester (Octocrylene; Eusolex®OCR, Neo Heliopan®Type 303, Uvinul®N 539 SG), o-aminobenzoic acid menthyl ester (Menthyl Anthranilate; Neo Heliopan®MA), 2,2′,4,4′-tetrahydroxybenzophenone (Benzophenone-2; Uvinul®D-50), 2,2′-dihydroxy-4,4′-dimethoxybenzophenone (Benzophenone-6), 2,2′-dihydroxy-4,4′-dimethoxybenzophenone-5-sodium sulfonate and 2-cyano-3,3-diphenylacrylic acid-2′-ethylhexyl ester. 4-Aminobenzoic acid, N,N,N-trimethyl-4-(2-oxoborn-3-ylidene methyl)aniline methyl sulfate, 3,3,5-trimethyl cyclohexyl salicylate, 2-hydroxy-4-methoxybenzophenone, 2-phenylbenzimidazole-5-sulfonic acid and potassium, sodium and triethanolamine salts thereof, 3,3′-(1,4-phenylenedimethylene)-bis(7,7-dimethyl-2-oxobicyclo-[2.2.1]hept-1-yl-methanesulfonic acid) and salts thereof, 1-(4-tert-butylphenyl)-3-(4-methoxyphenyl)propane-1,3-dione, α-(2-oxobom-3-ylidene)toluene-4-sulfonic acid and salts thereof, ethoxylated 4-aminobenzoic acid ethyl ester, 4-dimethylaminobenzoic acid-2-ethylhexyl ester, salicylic acid-2-ethylhexyl ester, 4-methoxycinnamic acid isopentyl ester, 4-methoxycinnamic acid-2-ethylhexyl ester, 2-hydroxy-4-methoxybenzophenone-5-sulfonic acid and the sodium salt thereof, 3-(4′-methylbenzylidene)-D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-yl acrylic acid and ethyl esters thereof, polymers of N-{(2 and 4)-[2-oxoborn-3-ylidene methyl]benzyl}acrylamide are preferred. Most particularly preferred according to the invention are 2-hydroxy-4-methoxybenzophenone, 2-phenylbenzimidazole-5-sulfonic acid and potassium, sodium and triethanolamine salts thereof, 1-(4-tert-butylphenyl)-3-(4-methoxyphenyl)propane-1,3-dione, 4-methoxycinnamic acid-2-ethylhexyl ester and 3-(4′-methylbenzylidene)-D,L-camphor. UV filters, whose molar extinction coefficient at the absorption maximum is above 15,000, in particular above 20,000, are preferred. It has moreover been found that with structurally similar UV filters, the non-water-soluble compound has in many cases the greater effect in the context of the teaching according to the invention as compared with water-soluble compounds that differ by one or more additional ionic groups. Within the context of the invention non-water-soluble is understood to mean UV filters that dissolve in water at 20° C. by no more than 1 wt. %, in particular no more than 0.1 wt. %. These compounds should furthermore be soluble in conventional cosmetic oil components at room temperature by at least 0.1, in particular at least 1 wt. %. The use of non-water-soluble UV filters can therefore be preferred according to the invention. According to a further embodiment of the invention UV filters having a cationic group, in particular a quaternary ammonium group, are preferred. These UV filters have the general structure U-Q.

The structural part U denotes a group that absorbs UV radiation. This group can in principle be derived from the aforementioned known UV filters that are suitable for use in the cosmetic sector by substituting a group, generally a hydrogen atom, of the UV filter with a cationic group Q, in particular having a quaternary amino function.

Compounds that can be derived from the structural part U are for example substituted benzophenones, p-aminobenzoic acid esters, diphenyl acrylic acid esters, cinnamic acid esters, salicylic acid esters, benzimidazoles and o-aminobenzoic acid esters.

Structural parts U that derive from cinnamic acid amide or from N,N-dimethylaminobenzoic acid amide are preferred according to the invention.

The structural parts U can in principle be chosen such that the absorption maximum of the UV filters can lie in both the UVA range (315-400 nm) and in the UVB range (280-315 nm) or the UVC range (<280 nm). UV filters having an absorption maximum in the UVB range, in particular in the range from approximately 280 to approximately 300 nm, are particularly preferred.

Depending also on the structural part Q, the structural part U is preferably chosen such that the molar extinction coefficient of the UV filter at the absorption maximum is above 15,000, in particular above 20,000.

The structural part Q preferably contains a quaternary ammonium group as the cationic group. This quaternary ammonium group can in principle be linked directly to the structural part U, such that the structural part U is one of the four substituents of the positively charged nitrogen atom. However, one of the four substituents at the positively charged nitrogen atom is preferably a group, in particular an alkylene group having 2 to 6 carbon atoms, that functions as a link between the structural part U and the positively charged nitrogen atom.

The group Q advantageously has the general structure —(CH₂)_{X}—N⁺R¹R²R³X⁻, in which x denotes a whole number from 1 to 4, R¹ and R² independently of each other denote C₁₋₄ alkyl groups, R³ denotes a C₁₋₂₂ alkyl group or a benzyl group and X⁻ denotes a physiologically tolerable anion. In the context of this general structure x preferably denotes the number 3, R¹ and R² each denote a methyl group and R³ denotes either a methyl group or a saturated or unsaturated, linear or branched hydrocarbon chain having 8 to 22, in particular 10 to 18, carbon atoms.

Physiologically tolerable anions are for example inorganic anions such as halides, in particular chloride, bromide and fluoride, sulfate ions and phosphate ions as well as organic anions such as lactate, citrate, acetate, tartrate, methosulfate and tosylate.

Two preferred UV filters having cationic groups are the compounds cinnamic acid amidopropyl trimethylammonium chloride (Incroquat®UV-283) and dodecyl dimethylaminobenzamidopropyl dimethylammonium tosylate (Escalol® HP 610), which are available as commercial products.

The present invention also comprises the use of a plurality of UV filters in combination. In the context of this embodiment the combination of at least one non-water-soluble UV filter with at least one UV filter having a cationic group is preferred. The UV filters (I) are conventionally contained in the agents used according to the invention in amounts from 0.1 to 5 wt. %, relative to the complete agent. Amounts from 0.4 to 2.5 wt. % are preferred. In the agents used according to the invention the UV filters improve the results of the repigmentation process, in the long term in particular, and are therefore particularly suitable. At least one of the aforementioned UV filters is particularly preferably combined with dihydroquercetin and at least one amino acid selected from taurine, proline, valine, arginine, lysine, and glycine.

It is also preferred to include additional polymers beyond those polymers from the group of cationic and/or amphoteric polymers. Preferred polymers, and the amounts in which they are contained in the compositions used according to the invention, are disclosed in DE 10 2009 044 974 on pages 43 to 45, incorporated herein by reference.

The agents used according to the invention may also comprise a 2-pyrrolidinone-5-carboxylic acid, and/or derivatives thereof. The sodium, potassium, calcium, magnesium or ammonium salts are preferred, in which the ammonium ion bears one to three C₁ to C₄ alkyl groups in addition to hydrogen. The sodium salt is most particularly preferred. The amounts used in the agents used according to the invention are preferably 0.05 to 10 wt. %, relative to the complete agent, particularly preferably 0.1 to 5, and in particular 0.1 to 3 wt. %.

Lastly, the agents used according to the invention may comprise plant extracts. These extracts are conventionally produced by extraction of the entire plant. It may also be preferable in individual cases, however, to produce the extracts exclusively from flowers and/or leaves of the plant. Useful extracts are listed in the table beginning on page 44 of the 3^{rd} edition of the Leitfaden zur Inhaltsstoffdeklaration kosmetischer Mittel, published by the Industrieverband Körperpflege- and Waschmittel e.V. (IKW), Frankfurt.

The extracts from green tea, oak bark, stinging nettle, witch hazel, hops, henna, chamomile, burdock, horsetail, whitethorn, lime blossom, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, meristem, ginseng and ginger root are preferred above all according to the invention.

The extracts from green tea, oak bark, stinging nettle, witch hazel, hops, chamomile, burdock, horsetail, lime blossom, almond, aloe vera, coconut, mango, apricot, lemon, wheat, kiwi, melon, orange, grapefruit, sage, rosemary, birch, lady's smock, wild thyme, yarrow, restharrow, meristem, ginseng and ginger root are particularly preferred.

The extracts from green tea, almond, aloe vera, coconut, mango, apricot, lemon, wheat, kiwi and melon are most particularly suitable for the use according to the invention.

Water, alcohols, and mixtures thereof, can be used as extracting agents to produce the cited plant extracts. Of the alcohols, lower alcohols such as ethanol and isopropanol, and also the polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

The plant extracts can be used according to the invention in both pure and diluted form. If they are used in diluted form they conventionally contain approximately 2 to 80 wt % of active substance and as the solvent the extracting agent or mixture of extracting agents used to obtain them.

It can furthermore be preferable to use mixtures of a plurality of different plant extracts, in particular two, in the agents used according to the invention.

It may be advantageous if penetration auxiliaries and/or swelling agents are contained in the agents used according to the invention. They include for example urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as for example 1,2-propanediol, 1,2-pentanediol, 1,2-hexanediol, 1,2-dodecanediol, 1,3-propanediol, 1,6-hexanediol, 1,5-pentanediol, 1,4-butanediol.

The present invention also comprises a method for positively influencing the natural pigmentation process of skin and/or skin appendages, in particular for stimulating the natural pigmentation process, in particular melanogenesis and/or pigmentation of the hair, for preventing and/or reducing graying of the hair and/or for repigmenting gray hair, wherein a combination of dihydroquercetin and/or a dihydroquercetin derivative with at least one amino acid is brought into contact topically with hair and/or skin.

All that has been stated in respect of the uses according to the invention applies with necessary alterations to further preferred embodiments of the method according to the invention.

### EXAMPLES

### Example 1

### Proof of the Differential Expression of Melanogenesis-Relevant Genes

The ligands involved in melanogenesis, such as SCF or alpha-MSH (melanocyte stimulating hormone alpha) bind to different receptors, through which the corresponding signal is transmitted into the cell interior. The receptor for SCF is ckit, the receptor for alpha-MSH is MCR-1 (melanocortin receptor 1). Substances that bring about a change in the expression of MCR-1 and/or ckit can influence melanogenesis. If an induction (up-regulation or stimulation) of the gene expression of the corresponding receptors occurs, melanogenesis is assumed to be stimulated.

Gp 100 is a protein that occurs in the membrane of melanosomes and stabilizes them. Since more melanin is produced in the cells following application of substances that positively influence melanogenesis, an increase in the melanosomes necessary for transport also occurs. A substance that induces the gene expression of gp100 is therefore a pigmentation-stimulating active ingredient.

Particularly preferred substances that stimulate the natural pigmentation process of skin and/or skin appendages, and in particular hair or hair follicles, are those that both bring about the gene expression of MCR-1 and/or ckit and induce the gene expression of gp100.

Determining the extent of the change in gene expression following an application of such substances to suitable cells/cell systems/tissue cultures can provide evidence of the effectiveness of the active ingredient.

Differential gene expression was determined by means of quantitative RT-PCR. After preparing three-dimensional organotypical hair follicle cell cultures from dermal papilla cells on microcarriers, they were incubated for 48 h with dihydroquercetin in two different concentrations. In order to perform PCR the RNA was first isolated from the organotypical cell cultures with the aid of an RNeasy Mini Kit from Qiagen and transcribed into cDNA by reverse transcription. In the subsequent PCR reaction, which is performed for each gene with the aid of gene-specific primers and which serves to amplify the required gene sections, the formation of PCR products is detected online via a fluorescence signal. The fluorescence signal is proportional to the amount of PCR product formed. The stronger the expression of a particular gene, the greater the amount of PCR product formed and the higher the fluorescence signal.

To quantify the gene expression the untreated control is set to 1 and the expression of the gene to be determined is referenced thereto (x-times expression). Values greater than or equal to the 1.8 times expression or less than or equal to the 0.5 times expression of the untreated control are classed as being expressed in a significantly differential manner. Values greater than or equal to the 1.5 times expression or less than or equal to the 0.7 times expression of the untreated control are classed as being expressed in a tangentially differential manner. The influence of dihydroquercetin on the expression of melanogenesis-regulating genes is shown in TABLE 1 below.

TABLE 1
Influence of dihydroquercetin on the expression of melanogenesis-
regulating genes.

| | Conc | ckit | | MCR1 | | gp100 | |
|---|---|---|---|---|---|---|---|
| Treatment | [μM] | Mean | SD | Mean | SD | Mean | SD |
| Untreated (Control) | — | 1.00 | 0.19 | 1.00 | 0.34 | 1.00 | 0.53 |
| Dihydroquercetin | 10 | 1.32 | 0.39 | 2.56 | 0.44 | 0.89 | 0.33 |
| Dihydroquercetin | 100 | 3.55 | 0.78 | 5.17 | 2.14 | 3.48 | 2.43 |

As shown, the expression of all three genes was induced in the 100 μM dihydroquercetin concentration. Even at a dihydroquercetin concentration of 10 μM, melanocortin receptor 1 was expressed in a significantly differential manner.

### Example 2

### Stimulation of Melanin Synthesis

Melanin is a dye that is produced and stored in the melanosomes of the melanocytes. Melanin gives the hair its intrinsic color, the coloration being formed by a mixture of two types of melanin, namely eumelanin and pheomelanin. Melanogenesis is a complicated and highly regulated synthesis process. Tyrosine is converted first by the enzyme tyrosinase into L-dihydroxyphenylalanine (L-DOPA) and then via a plurality of intermediate steps into the various melanin pigments. An active ingredient that positively influences melanogenesis and leads to an increased melanin content in the hair follicle melanocytes is particularly suitable for influencing the natural pigmentation process of skin and/or skin appendages, preventing hair graying and/or stimulating pigmentation.

In order to assess the melanin content, three-dimensional organotypical hair follicle cell cultures prepared from dermal papilla cells, hair follicle melanocytes and outer root sheath keratinocytes were treated with dihydroquercetin (10 μM and 100 μM) for 7 days. Untreated cell cultures served as a control. After 4 and 7 days respectively, the hair follicle equivalents were homogenized and the melanin extracted with NaOH (1M)+10% DMSO at 100° C. for 45 min. Aliquots of the specimens were then transferred to a 96-well plate and the extinction was measured at 492 nm. The rise in the melanin content from day 0 to day 7 was evaluated. All values were referenced to the untreated control on day 7 (=100%). As shown below in TABLE 2, melanin synthesis in the treated cultures was able to be increased markedly in comparison to the control at both concentration levels tested.

TABLE 2
Melanin content in hair follicle equivalents after treatment with
dihydroquercetin.

| | | Melanin content [%] | | | |
|---|---|---|---|---|---|
| | | d4 | | d7 | |
| Treatment | Conc [μM] | Mean | SD | Mean | SD |
| Untreated (Control) | — | 100 | 28 | 181 | 86 |
| Dihydroquercetin | 10 | 100 | 28 | 254 | 58 |
| Dihydroquercetin | 100 | 100 | 28 | 161 | 22 |

### Example 3

### Effect of a Mixture of Taurine, Proline, Valine, Arginine, Lysine and Glycine

A mixture of taurine, proline, valine, arginine, lysine and glycine (1:1:1:1:1 ratio, stated in relation to the proportions in the complete mixture) was examined in respect of its effects on ATP synthesis and release of hepatocyte growth factor (HGF). ATP (adenosine triphosphate) is the universal storage form for chemical energy in cells. Cleaving off the phosphate groups produces ADP and Pi (inorganic phosphate). This reaction is highly exergonic, in other words energy is released. ATP is produced in the cellular, oxidative breakdown of fats, carbohydrates and proteins. It serves as an energy source for biochemical synthesis (also melanin synthesis), for transport processes (active transport) and for mechanical work. These processes are endergonic, in other words they proceed only with an input of energy. HGF is an important growth factor, with the aid of which the dermal papilla controls hair growth and the hair cycle to which pigment production in the hair follicle is particularly linked. Melanin formation takes place exclusively in the anagen phase of the hair cycle. Furthermore, the effect of HGF on DNA synthesis and on the growth and differentiation of melanocytes is discussed in various publications.

ATP determination took place using the ATPLite™-M assay (Packard). The test principle of this assay is based on the fact that the luciferase of Photinus pyralis catalyzes a reaction in which D-luciferin is converted into oxyluciferin in the presence of ATP. In this reaction green light is emitted that can be measured with a luminometer. The emitted bioluminescent light is proportional to the amount of ATP present.

The ATP activity was determined in organotypical cell cultures prepared from three-dimensionally cultivated dermal papilla cells. Treatment with the mixture of substances took place over 24 hours in comparison with an untreated control. The results are shown in TABLE 3 below.

TABLE 3
ATP content in hair follicle cell cultures after treatment with the
amino acid mixture taurine, proline, valine, arginine, lysine,
and glycine (1:1:1:1:1:1).

ATP [%]

| Treatment | Conc [%] | Mean | SD |
|---|---|---|---|
| Untreated (Control) | — | 100 | 23 |
| Amino acid mixture (1:1:1:1:1:1) | 0.001 | 130 | 15 |
| Amino acid mixture (1:1:1:1:1:1) | 0.005 | 130 | 22 |
| Amino acid mixture (1:1:1:1:1:1) | 0.01 | 136 | 23 |

As shown in TABLE 3, the ATP content in the treated cultures was increased markedly in comparison to the control for each of the concentrations tested.

The release of HGF can be quantified with the aid of a commercially available ELISA kit. To this end, organotypical hair follicle cell cultures prepared from dermal papilla cells, hair follicle melanocytes, and outer root sheath keratinocytes were incubated with the preferred six-amino acid mixture (as in TABLE 3) for 72 h and the concentration of HGF in the medium was determined. The percent (%) HGF released is shown in TABLE 4 below.

TABLE 4
Relative release of HGF in [%].

HGF [%]

| Treatment | Conc [%] | Mean | SD |
|---|---|---|---|
| Untreated | — | 100 | 33 |
| Amino acid mixture (1:1:1:1:1:1) | 0.001 | 220 | 32 |
| Amino acid mixture (1:1:1:1:1:1) | 0.005 | 274 | 29 |
| Amino acid mixture (1:1:1:1:1:1) | 0.01% | 151 | 30 |

For the 0.001% and 0.005% usage concentrations tested, the HGF content in the treated cultures was increased significantly compared to the untreated control.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. A hair treatment agent comprising:
a. from 0.000001 to 3 wt. % of a flavonoid selected from the group consisting of dihydroquercetin, dihydroquercetin derivatives, and mixtures thereof; and
b. from 0.000001 to 5 wt. % of at least one amino acid.

2. The hair treatment agent of claim 1, wherein said amino acid is chosen from the group consisting of taurine, proline, valine, arginine, lysine, glycine, and mixtures thereof.

3. The hair treatment agent of claim 1, wherein said dihydroquercetin derivative is chosen from the group consisting of dihydroquercetin monomethyl ethers, dihydroquercetin dimethyl ethers, dihydroquercetin glycosides, dihydroquercetin glucosides, dihydroquercetin xylosides, dihydroquercetin rhamnosides, dihydroquercetin galacto sides, and mixtures thereof.

4. The hair treatment agent of claim 1, wherein said at least one amino acid is a six amino acid mixture consisting essentially of a 1:1:1:1:1:1 ratio of taurine, proline, valine, arginine, lysine, and glycine.

5. The hair treatment agent of claim 1, wherein the ratio of said flavonoid to said at least one amino acid is from 10:1 to 1:10.

6. The hair treatment agent of claim 5, wherein said ratio is from 1:2 to 2:1.

7. The hair treatment agent of claim 1, further comprising a tocopherol.

8. The hair treatment agent of claim 7, wherein said tocopherol is α-tocopherol.

9. The hair treatment agent of claim 1, further comprising at least one gelling agent.

10. The hair treatment agent of claim 1, further comprising at least one monohydric alcohol chosen from the group consisting of ethanol, n-propanol, isopropanol, n-butanol, and mixtures thereof.

11. A hair treatment agent comprising:
a. from 0.1 to 90 wt. % of at least one monohydric alcohol chosen from the group consisting of ethanol, n-propanol, isopropanol, n-butanol, and mixtures thereof;
b. from 0.000001 to 3 wt. % of a flavonoid selected from the group consisting of dihydroquercetin, dihydroquercetin derivatives, and mixtures thereof;
c. from 0.000001 to 5 wt. % of at least one amino acid selected from the group consisting of taurine, proline, valine, arginine, lysine, glycine, and mixtures thereof; and
d. optionally, from 0 to 10 wt. % of at least one gelling agent.

12. A method for stimulating the natural pigmentation process of hair, said method comprising the step of:
a. topically contacting hair with the hair treatment agent of claim 1.

13. A method for reducing graying of hair or for repigmenting gray hair, said method comprising the step of:
a. topically contacting hair with the hair treatment agent of claim 1.

14. A method for stimulating melanogenesis in hair follicles, said method comprising the step of:
a. topically contacting hair follicles with the hair treatment agent of claim 1.
